(19) United States
(12) Patent Application Publication
(10) Pub. No.: US 2003/0219754 A1
(43) Pub. Date: Nov. 27, 2003

(54) Fluorescence polarization detection of nucleic acids

(76) Inventors: Jerome E. Oleksy, Park Ridge, IL (US); Faye A. Boeckman, Elmhurst, IL (US); Eric A. Hungate, Des Plaines, IL (US); Erik V. Rencs, Ellicott City, MD (US)

Correspondence Address: FISH & RICHARDSON PC, 225 FRANKLIN ST, BOSTON, MA, 02110, US

(21) Appl. No.: 10/155,285

(22) Filed: May 23, 2002

(51) Int. Cl. C12Q 1/68; C12M 1/34

(52) U.S. Cl. 435/6; 435/287.2

(57) ABSTRACT

The apparatus and method described herein detect fluorescence polarization (FP) during a nucleic acid reaction such as PCR amplification or isothermal amplification. Fluorescence polarization can be concurrently detected in multiple samples. In addition, multiple different fluorophores can be used for detect different sequences within a sample during the same reaction.

### BACKGROUND

This description relates to nucleic acid detection by fluorescence polarization.

The polymerase chain reaction (PCR) can be used to detect small quantities of specific nucleic acids in a sample. So-called real-time PCR monitors nucleic acid in a PCR reaction during the course of the reaction. Real-time PCR has aided quantitation of nucleic acid concentrations.

Higuchi R et al, (Biotechnology (NY) 1993;11(9): 1026-1030) described a real-time PCR method that used the intercalating dye, ethidium bromide to monitor the amount of amplified nucleic acid present during the reaction. The fluorescence of ethidium bromide is altered when the dye intercalates. The level of fluorescence during the reaction was plotted against time and used to determine the amount of starting sample. Quantitation of test samples was achieved with high sensitivity and reliability. However, the binding of ethidium bromide to nucleic acid is not specific to a particular target sequence and, thus, cannot discriminate between target sequences and non-target sequences.

Another real-time PCR technique includes a 5′ nuclease assay in which a probe sequence specific for the target sequence is monitored (Holland P M et al, (1991) Proc. Natl. Acad. Sci. USA 88: 7276-7280; Lee L G et al, (1993) Nucl Acids Res 21(16): 3761-3766; Livak K J et al, (1995) PCR Meth. Appl. 4(6): 357-362). The probe in this assay includes a 5′ fluorescent dye and a 3′ quenching dye. When the probe binds to a template, the 5′ exonuclease activity of the DNA polymerase cleaves the 3′ quenching dye from the primer. The 5′ fluorescent dye then provides a stronger fluorescent signal when excited.

### SUMMARY

In one aspect, the invention features an apparatus that includes a light source to concurrently excite fluorescent compounds, located in a plurality of spatially distinguishable areas within a first region of a sample carrier, with polarized light; and a detection system to concurrently detect emitted light from the fluorescent compounds in each of the areas of the plurality in the first region. The apparatus can further include a thermal control unit to regulate the temperature of a sample carrier that includes spatially distinct nucleic acid samples, e.g., to cyclically heat and cool the carrier during a reaction course.

The detection system can be further configured to concurrently detect emitted light at at least a plurality of instances during the reaction course. Further, the detection system may detect emitted light from the first region without movement of the sample carrier relative to one or both of the detection system and the light source.

The apparatus can further include the sample carrier, e.g., a carrier having a plurality of discrete areas. For example, each area of the plurality includes a container within the sample carrier. Each container can include one of the physically distinct nucleic acid samples. In one embodiment, the first region can include all physically distinct samples of the sample carrier.

In one embodiment, at least a subset of the discrete areas of the plurality are continuous with each other (e.g., not physically isolated from each other). Each area of the plurality of discrete areas can include a nucleic acid polymerase (e.g., an RNA or DNA polymerase) and/or a nucleic acid ligase. Exemplary samples include a synthetic or biological sample, such as a histological preparation, a cell, an extract or a cell, an environmental sample. In the case of a cellular sample, the cell can be spread.

In one embodiment, the detection system is further configured to detect emitted light of a first polarity and emitted light of a second polarity.

The first and second polarities can be approximately orthogonal to each other. In one embodiment, the first and second polarities are at least 30° apart, e.g., about 45° apart. In one embodiment, the first polarity is approximately (e.g., within 10°) parallel to the polarity of the polarized light from the light source. The second polarity can be non-parallel (e.g., perpendicular) to the polarity of the polarized light from the light source. The detection system can be further configured to detect emitted light of at least a third polarity.

The detection system can detect the first and second polarity light concurrently. In one embodiment, the detection system includes a first and second detector. In another embodiment, the detection system consists of a single detector, and the first and second polarity light are projected onto different regions of the detector.

The detection system can detect the first and second polarity light sequentially. In one embodiment, the detection system includes a polarizer that is controlled to enable detection of the first and second polarity light.

The detection system and the light source can be in a signal communication, e.g., to enable transient-state detection, e.g., wherein detection of emitted light is temporally delayed relative to the excitation. The apparatus may also be configured for steady-state detection.

In one embodiment, the detection system is further configured to distinguish polarized light from a first fluorophore from polarized light of at least a second fluorophore, e.g., to detect and distinguish fluorescence polarization of a first fluorophore and fluorescence polarization of a second fluorophore. The system may further detect and/or distinguish fluorescence polarization of a third fluorophore (e.g., greater than five or six fluorophore), e.g., a fluorophore described herein.

The thermal control unit can further include a thermal probe that detects solution temperature in a sample of the sample carrier and/or a heat source and heat sink. In one embodiment, the thermal control unit is further configured to selectively apply a thermal gradient to the sample carrier. The thermal control unit may also be regulated by a processor that can receive (directly or indirectly) instructions provided by a user, e.g., from a user interface. The instructions may include information for thermal cycling.

The light source can include a bulb or a laser. The light source can include one or more of a band-pass filter, polarizer, and diffuser. The light source can be positioned to illuminate one or more optical fibers. In one embodiment, the apparatus includes a plurality of optical fiber bundles, including one bundle configured to illuminate a first plurality of regions of the sample carrier and a second bundle configure a second plurality of regions of the sample carrier. The regions of the first and second plurality may overlap, e.g., the first and second plurality of regions may be co-extensive. For example, the first plurality of regions may correspond to regions spaced by a first index, while the second plurality of regions may correspond to regions spaced at a second index.

In one embodiment, the apparatus further includes a beam splitter, positioned to reflect excitation light to the sample carrier and/or emitted light to the detector, respectively. In one embodiment, the apparatus further includes a scanning mirror, e.g., a scanning mirror positioned to reflect excitation light from a light source or emitted light to a detector.

The detection system can include a photo-multiplier tube (PMT) or a charged coupled device (CCD). The detection system can include an imaging system that generates an image map (e.g., a pixilated image).

In one embodiment, the light path emanating from the light source is parallel to the incident light path into the detector. In one embodiment, the detector is positioned between (or in-line) the light source and an imageable surface of the sample carrier.

In another embodiment, light path from the light source to the sample carrier surface or the light path from an imageable surface of the sample carrier to the detector is oblique with respect to the imageable surface. For example, both light paths can be oblique.

In one embodiment, the light source is further configured to excite a second region and the detection system is further configured to detect emitted light from the second region.

In another aspect, the invention features an apparatus that includes: a plurality of spatially distinguishable reaction samples, each including amplification reagents that include a nucleic acid primer that is attached to a fluorophore; an amplification control unit that is configured to control conditions of the reaction samples for nucleic acid amplification; and a fluorescence polarization monitor that is configured to concurrently monitor fluorescence polarization associated with each reaction sample of the plurality. Embodiments of the apparatus can include any feature described herein. The apparatus can also include a second plurality of samples.

In still another aspect, the invention features a method that includes: providing a plurality of spatially distinct nucleic acid samples and amplification reagents that includes a fluorophore attached to a nucleic acid primer; concurrently amplifying each sample of the plurality; and, during the amplifying, concurrently detecting fluorescence polarization information associated with the fluorophore from each sample of the plurality. Each primer can be specific for a different nucleic acid species.

Fluorescence polarization information at least includes information that relates to the amount of emitted light in a plane parallel to the plane of excitation light. In some cases, fluorescence polarization information includes information that relates to the amount of emitted light in a plane parallel to the plane of excitation light and the amount of emitted light in a plane perpendicular to the plane of excitation light. It may be convenient to express fluorescence polarization information as a value that is a function of both the amount of emitted light in a plane parallel to the plane of excitation light and that relates to the amount of emitted light in a plane perpendicular.

The detecting can include detecting fluorescence polarization information at at least a plurality of instances during the amplifying. In one embodiment, the instances are at regular intervals, e.g., at regular intervals until amplification of at least some samples reaches saturation phase. In one embodiment, the amplifying includes thermal cycles and the detecting includes detecting fluorescence polarization information at at least one instance for each cycle.

In one embodiment, the amplifying and detecting are effected by an apparatus described herein, e.g., an apparatus that includes: a light source configured to concurrently excite the fluorophores, located in a plurality of the spatially distinct samples, with polarized light; and a detection system configured to concurrently detect emitted light from the fluorophores in each of the spatially distinct samples of the plurality.

Both exponential and linear amplification methods can be used. In one embodiment, the amplifying depends on DNA polymerase activity, e.g., a thermal stable DNA polymerase activity. For example, the amplifying can include thermal cycles, e.g., PCR amplification, e.g., exponential or linear PCR amplification (e.g., without a second primer). In another embodiment, the amplifying depends on RNA polymerase activity. For example, the amplifying is isothermal. In still another embodiment, the amplifying comprises a sequence specific cleavage event, e.g., endonucleolytic cleavage of a flap. Each of the samples can be disposed at a separate address of a sample carrier. For example, the sample carrier can include a multi-well plate, a planar array, and so forth. In one embodiment, the sample carrier is not uniformly heated.

In one embodiment, the detecting includes detecting emitted light of a first and second polarity. The light of first and second polarity can be detected concurrently or at separate times.

The method can further include other features or aspects described herein, e.g., a feature associated with use of an apparatus described herein.

In another aspect, the invention features a method that includes: providing a sample carrier that includes a plurality of reaction mixtures, each mixture including a nucleic acid sample and amplification reagents that includes a first fluorophore attached to a first nucleic acid primer and a second fluorophore attached to a second nucleic acid primer; amplifying target nucleic acid, if present, in each of the reaction mixtures the sample using the first and second primers; and at at least a plurality of instances during the amplifying, detecting fluorescence polarization information associated with the fluorophore, wherein the sample carrier is stationary throughout the amplifying. In one embodiment, the mixture includes at least a third, fourth, fifth, and sixth fluorophore, each attached to a primer. The fluorophores can be spectrally resolved from each other, e.g., in the excitation or emission channel, or both. Exemplary fluorophores include: fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. The method can further include other features or aspects described herein, e.g., a feature associated with use of an apparatus described herein.

In still another aspect, the invention features a method that includes: providing a reaction mixture that include a nucleic acid sample, amplification reagents, and a fluorescent probe that is bindable to double-stranded nucleic acid; amplifying each sample of the plurality; and during the amplifying, detecting fluorescence polarization information associated with the fluorescent probe at at least a plurality of instances. The fluorescent probe can have at least a 10, 20, 50, 100, 200 or 400-fold preference for binding double-stranded nucleic acid relative to single-stranded nucleic acid. In one embodiment, the fluorescent probe is an intercalating dye, e.g., Sybr Green or ethidium bromide. In one embodiment, a plurality of reaction mixtures having different nucleic acid samples are provided, and the mixtures are concurrently amplified and concurrently detected. The method can further include other features or aspects described herein, e.g., a feature associated with use of an apparatus described herein.

In another aspect, the invention features a method of multiplex nucleic acid analysis. The method includes: providing a nucleic acid sample and amplification reagents that includes a first fluorophore attached to a first nucleic acid primer and a second fluorophore attached to a second nucleic acid primer; amplifying nucleic acid in the sample using the first and second primers; and at at least a plurality of instances during the amplifying, detecting fluorescence polarization information associated with each of the fluorophores.

In one embodiment, the first and second fluorophore have distinguishable absorption and/or emission spectra. The emission spectra can be, in some cases, partially overlapping. The detecting can include sequentially detecting fluorescence polarization information of the first fluorophore at a first wavelength and information from the second fluorophore at a second wavelength.

In one embodiment, the first and second primers hybridize to different genes. In another embodiment, the first and second primers hybridize to the same gene, e.g., different alleles of the same gene, different splicing variants of the same gene, or different regions of the same gene. For example, the first and second primer can be partially overlapping.

The first and second primer can hybridize differentially to an allele of a polymorphism, e.g., a single nucleotide polymorphism. In one embodiment, the first primer includes a region that has fewer mismatches when hybridized to a first allele of a polymorphism than to a second allele of the polymorphism. For example, the region of the first primer can be exactly complementary to a first allele of a polymorphism and partially complementary to the second allele of the polymorphism. In a related example, the region includes at least one position that is a mismatch when hybridized to the first and also when hybridized to the second allele. In another example, the region includes a mismatched position when hybridized to the second allele, but not the first allele. The mismatched position can be at any position within the primer, for example, in the center of primer, or within 4, 3, 2, or 1 nucleotides of the 3′ end. In one embodiment, the mismatched position is at the 3′ end. Similarly, the second primer can include a region that has fewer mismatches when hybridized to the second allele of a polymorphism than to the first second allele of the polymorphism. In one embodiment, the first and second primer have the same length in nucleotides. The method can further include other features or aspects described herein, e.g., a feature associated with use of an apparatus described herein.

In another aspect, the invention features an article of machine-readable medium, having embodied thereon instructions that cause a processor to effect a method of analyzing fluorescence information, e.g., fluorescence polarization information. The method includes: receiving fluorescence information (e.g., fluorescence polarization values and/or fluorescence intensity values), each instance of information being associated with an temporal instance during a nucleic acid amplification reaction; extrapolating an initial value from intensity values within an exponential region of the amplification reaction; and inferring an initial concentration for the target nucleic acid. The method can include analyzing intensity values from a reference sample, e.g., a sample of known nucleic acid concentration for a given sequence composition. In one embodiment, a single reference sample is used.

As seen above, fluorescence polarization information can include a value representing fluorescence approximately perpendicular and a value approximately parallel to polarized excitation light. The fluorescence information can be detected from a fluorophore attached to a primer specific for a target nucleic acid. The method can include effecting the display or transmittal of the inferred initial concentration. The method can also further include comparing of the inferred initial concentration for the target nucleic acid to a similarly inferred initial concentration for a reference nucleic acid. The extrapolating can include linearly extrapolating the logarithm of a fluorescence value against a temporal value.

In still another aspect, the invention features an article of machine-readable medium, having embodied thereon instructions that cause a processor to effect a method including: receiving a plurality of image maps, each map including information about detected light of a defined polarity at a plurality of imaged sites, each imaged site including a primer for amplification of a target nucleic acid; and determining a value indicative of abundance of extended primers at each of the imaged sites. Each image map can include a plurality of pixels for each of the imaged sites. The image map can include image information from a CCD, e.g., raw or processed image information. Each of the imaged sites can correspond to a sample on a multi-sample carrier.

In one embodiment, the plurality of image maps include maps including information about detected light of a first defined polarity and maps including information about detected light of a second defined polarity. For example, the first and the second defined polarities are orthogonal to each other.

The plurality of image maps can include maps including information about detected light at different instances during a reaction, e.g., instances occurring during different thermal cycles.

The method can further include inferring an initial concentration of target nucleic acid for each of the imaged sites from the determined values for each site. The target nucleic acids can differ among at least two of the imaged sites. The inferred concentrations can be associated with other information, e.g., information representing the identity of the target nucleic acid at a particular site. In one embodiment, at least a plurality of primers are present at each imaged site, and the primers of the plurality include different fluorophores with respect to other primers of the plurality. The information about detected light can include information that distinguishes the different fluorophores.

In still another aspect, the invention features a database, stored on machine-readable medium. The database includes data representing (a) fluorescence polarization assessments, (b) reaction samples, (c) temporal information; and associations that relate each fluorescence polarization assessment to a reaction sample and to a temporal value.

The fluorescence polarization assessments can include assessments of detected light of a first polarity and detected light of a second polarity. In one embodiment, the first and second polarity are orthogonal. The temporal values can correspond to times during an amplification reaction, e.g., different amplification cycles. The data can further represent (d) association with a given primer as well as other useful information.

In another aspect, the invention a database that includes a plurality of image maps, each map including information about detected light of a defined polarity at a plurality of imaged sites, wherein the detected light of each map is associated with a fluorophore and each map is associated with a temporal position during an nucleic acid amplification reaction. In one embodiment, the map is pixilated, and each image site corresponds to a plurality of pixels. In one embodiment, the plurality includes maps including information about detected light of a first defined polarity and maps including information about detected light of a second defined polarity. For example, each mapped value of the maps of the plurality is a function of detected light of a first defined polarity and detected light of a second defined polarity, the first and second polarity being orthogonal to each other. In one embodiment, the plurality includes maps including information about fluorescence polarization of a first fluorophore and maps including information about fluorescence polarization of a second fluorophore.

The invention also features a system that includes an apparatus described herein and a processor configured to receive information from the apparatus about the fluorescence polarization. For example, the apparatus includes (1) an amplification control unit that is configured to control reaction conditions at a plurality of sites for nucleic acid amplification; and (2) a fluorescence polarization monitor that is configured to concurrently monitor fluorescence polarization associated with each site of the plurality. The processor can be further configured to send instructions that control temperature with time, e.g., to effect thermal cycling. Other sent instructions can include a trigger to monitor fluorescence polarization.

The received information about the detected light can include an image or image map, e.g., for a pixilated image. In one embodiment, the received information includes an overall value for each sample for a given monitoring event.

The processor can also receive information about a reaction condition, e.g., information about detected temperature, e.g., periodically or continuously. In one embodiment, the processor receives information in bulk (e.g., at once for a plurality of monitoring events, e.g., temporally separate monitoring events.).

The processor can be further configured to infer concentrations of nucleic acid and/or to display, store, or transmit the inferred concentrations.

In one embodiment, the processor and the apparatus are in signal communication via a serial connection. In another embodiment, the processor and the apparatus are in signal communication via a computer network, e.g., a wireless network or an ethernet. In one embodiment, the system further includes a server in signal communication with the processor.

At least one advantage of the featured methods and apparati are that nucleic acid amplification can be monitored in multiple nucleic acid samples rapidly, and in some cases concurrently. An image of a collection of samples can be taken at intervals during an amplification reaction. Processing of the image can indicate the concentration of target nucleic acids in the initial sample. Rapid imaging not only enables more samples to be processed in a given time frame, but may also provide increased accuracy and reproducibility in the amplification process. Likewise, the use of only one labeled primer (the primer, itself, typically having only a single label) for a given target sequence, is economical. Further, it enables some implementation to detect amplification by multiple primers, each specific for a different target sequence.

Other features and advantages of the invention will be apparent from the following detailed description, and from the claims. All patents and references cited herein are incorporated in their entirety by reference.

### BRIEF DESCRIPTION OF THE DRAWINGS

FIGS. 1 to 10, and 13 to 15 are schematics of exemplary apparati.

FIG. 11 is a block diagram of an exemplary computer for operating software.

FIG. 12 is a schematic of an exemplary system.

### DETAILED DESCRIPTION

Fluorescence Polarization

The apparatus and methods described herein enable the detection of fluorescence polarization (FP) during a nucleic acid reaction such as PCR amplification or isothermal amplification. Typically, FP is concurrently detected in multiple samples.

FP measurements are a function of the size or molecular weight of a molecule since these parameters contribute to the molecule's rotational rate in solution. Specifically, the rotational rate varies inversely with size. FP can effectively discriminate between small and large molecules by virtue of its rotational rate if a fluorophore is attached to the molecule. Because larger molecules rotate more slowly, a larger component of their emitted fluorescence is light parallel to the plane of the excitation light. Accordingly, some FP measurements are made mostly of light parallel to the plane of excitation light (Fₚₐᵣₐₗₗₑₗ). Other FP measurements, of course, also include a measurement of light emitted in a plane non-parallel—typically, perpendicular—to the plane of excitation light (e.g., F_{perpendicular}). One standard FP value is the following ratio:

$$\frac{{F}_{Parallel} - {F}_{Perpendicular}}{{F}_{Parallel} + {F}_{Perpendicular}} ,$$

where F is a relative measure of light intensity (RFU, relative fluorescence units).

Other relationships that provide an FP value are also useful.

FP-PCR monitors the rotational rate of a fluorophore incorporated into a PCR primer. A labeled primer can have at least four apparent sizes:

1. Unextended and unhybridized (i.e., single-stranded)

2. Unextended and hybridized to a target;

3. Extended and unhybridized (i.e., single-stranded);

4. Extended and hybridized to a target.

Each of these forms has a different molecular size, and consequently a different FP value. FP measurements (e.g., Fₚₐᵣₐₗₗₑₗ and/or F_{Perpeindicular}) can be used to determine the amount of extended or unextended primer, and/or the amount of hybridized or unhybridized primer.

During a nucleic acid amplification reaction, primers are extended (e.g., by a polymerase or a ligase). As the primer is incorporated into a longer nucleic acid, its molecular weight and size increases. The longer nucleic acid has the correspondingly slower rotational rate of a larger molecule and increased FP value. FP can, thus, sensitively monitor the extension of a primer as the reaction proceeds (e.g., at instances during the reaction).

Annealing of the primer to a complementary nucleic acid strand can also be detected. When annealed to a complementary strand, the primer-complement complex has the rotational rate of a larger molecule and is consequently detected as such by FP. Thus, under conditions where the primer can anneal to its complement, the FP, likewise, provides a measure of both the concentration of the complementary strand and the amount of extended primer.

Depending on the implementation, conditions can be selected to control the extent of hybridization of the unextended primer. For example, FP measurements can be made at a temperature sufficiently below the Tm of the primer, in which case, if the product is present, the unextended primer is annealed. Likewise, at a temperature sufficiently above the Tₘ of the primer, unextended primer is not annealed. The primer can also be designed so that its Tₘ is, e.g., less than a predetermined value, e.g., a predetermined value in the range of 40 to 55, 50 to 60, or 47 to 55° C. In the example of PCR amplification, FP measurements can be made at least once per cycle, e.g., at the same predetermined temperature each cycle.

Regardless of the annealing state of the primers, as an amplification reaction proceeds, more primers are extended and incorporated into the product, and more product is produced, thus increasing the average polarization value of the sample.

The FP value can be correlated with the amount of nucleic acid product present. For example, data can be collected at each PCR cycle for real-time detection. The relationship between FP values during the PCR reaction provides useful information about the sample. A plot of FP values vs. PCR cycle (e.g., ln(FP) vs. PCR cycle) can be used to extrapolate the initial concentration of target nucleic acid in the sample prior to amplification.

Unlike some other real-time PCR methods, FP-PCR can be implemented with a single labeled primer having a single label (i.e., the fluorophore). Of course, other implementations, e.g., with multiple labels and multiple labeled primers are possible. Specificity of the PCR target is achieved by the design of sequence-specific primers, eliminating the need for a secondary probe to query the amplified nucleic acid.

Moreover, a number of alternative implementations can also be used. In one implementation, the labeled primer is diluted with an unlabeled primer with identical sequence. For example, ratio of labeled to unlabeled primer can be less than 1.0, 0.25, or 0.1. In another implementation, both primers of a PCR primer are unlabeled. Product is detected by a labeled oligonucleotide that is unextendable and which hybridizes to one of the product strands.

Apparatus for FP Analysis of PCR amplification

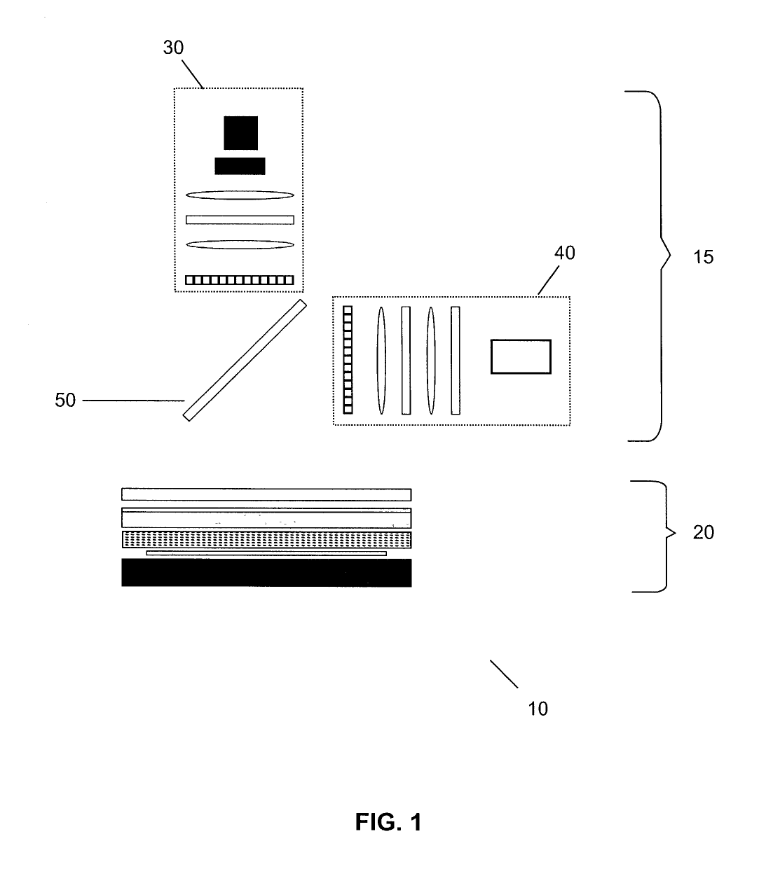

Referring to FIG. 1, a typical apparatus 10 for FP-PCR analysis includes an optical assembly 15 and a thermal cycler assembly 20.

Thermal Cycler Assembly

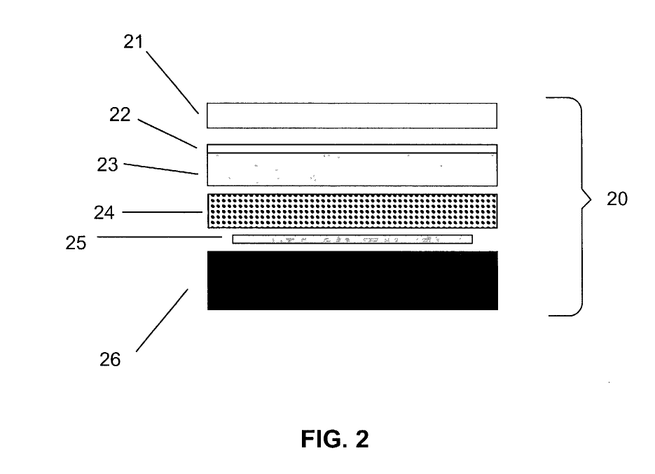

Referring to FIG. 2, the thermal cycler assembly 20 includes a heat transfer block 24 upon which a sample carrier 23 is disposed. The temperature of the heat transfer block 24 is controlled by a heat-cold source 25 and a heat sink 26 for cooling. Other designs can be provided by one of ordinary skill in the art. For example, the construction of Peltier-effect devices for PCR are known. These devices use a solid-state technology for thermoelectric heating and cooling. The devices can operate without moving parts, and usually has a fan to remove excess heat.

In some embodiments, the heat transfer block 24 is configured to provide a spatial temperature gradient.

The sample carrier 23 can include a plurality of areas on or in which reactions can occur, e.g., for replicates or different samples. Exemplary sample carriers include a microtitre plate, one or more (e.g., an array) of capillaries, a microfluidic system (e.g., cartridge) and so forth. For example, the sample carrier can include multiple containers such as the multiple wells of a standard microtitre plate with 96 or 384 wells. In another example, the sample carrier includes a histological sample for in situ amplification, e.g., the sample carrier includes a planar glass surface. In still another example, the carrier includes a set of arrayed samples on a contiguous surface.

The sample carrier 23 is covered by a transparent seal 22. For example, the seal can be composed of materials such as plastics that are transparent to visible and UV light, e.g., a material that is uniformly birefringent, e.g., a material such as polyester or polyolefin. The seal 22 is, in turn, covered by a transparent heated lid 21. The heated lid 21 can serve at least two functions. One function is to apply pressure to the seal so that it retains closure of the wells. A second function is to maintain the temperature on the top of the sample carrier 23 during PCR amplification, e.g., to prevent condensation of liquid that may evaporate from the sample. The heated lid 21 can be composed of common optical materials such as BK7 or Fused-Silica and may encompass a thin-film, optically transparent heat source or be attached to another type of heat source that provides the required temperature (e.g., ˜104° C.) and uniformity of temperature (e.g., ˜4° C.).

Referring again to FIG. 1, the optical assembly 15 includes a light source assembly 40 and a detector 30. The light source can project a beam of excitation light parallel to the surface of the thermal cycler assembly 20. A beam splitter 50 can be used to enable the excitation light to be reflected from the excitation source directly onto the upper surface of the thermal cycler assembly 20.

Light Source Module

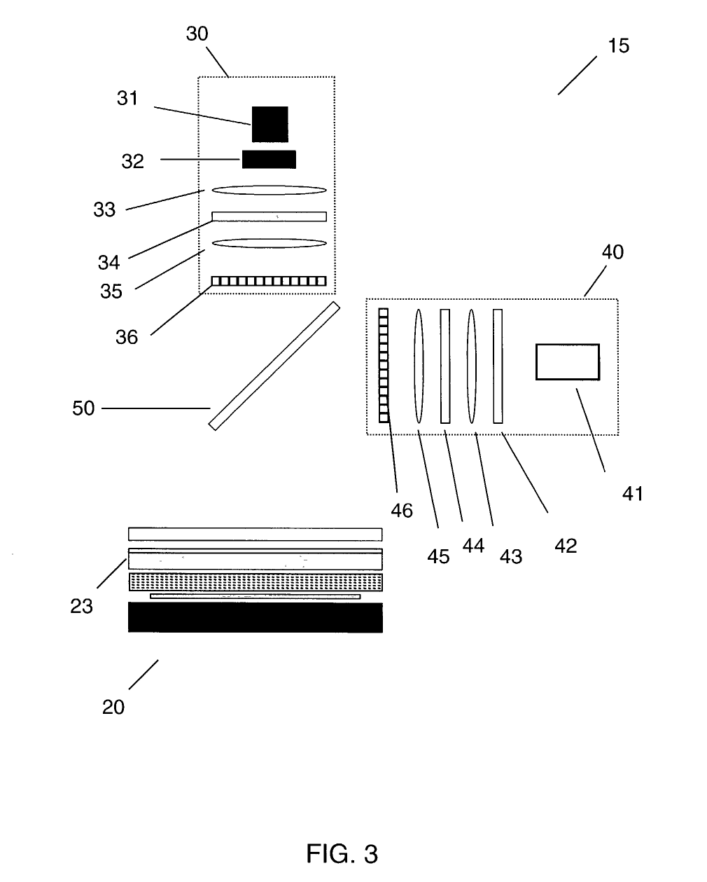

Referring also to FIG. 3, the light source assembly 40 includes a light source 41. Light from the source 41 passes through a heat-absorbing filter 42, then a lens 43, a band-pass filter 44, a second lens 45, and a polarizer 46. Of course, some components, such as the heat-absorbing filter 42, are optional; other components, not shown here, can also be included. Specifics depend on the implementation and the desired performance.

Light Source 41. The light source 41 has several important components. The source itself can be one of several configurations. The source 41 can be a laser, a quartz-tungsten halogen, a Xenon (continuous or flash) light source, a mercury light source and others. The source can be a bulb that emits in all directions and requires collecting and directing optics to make it efficient. Other sources can have optical components built into the design that collect and direct the light. If the source is a non-polarized source, then the light is subsequently polarized (e.g., see polarizers, below) to provide and limit the light that reaches the sample to one direction of linear polarization.

Linear excitation polarization can also be achieved by utilizing a polarized light source, such as a laser. In some implementations, broadband tunable lasers can be used as they have the capability of a wavelength tunable polarized source.

Depending on the implementation and desired performance, it may be advantageous to use a non-polarized source or to use a polarized source.

Band-Pass Filters 44. In the case of broadband sources, other optical components such as lenses direct light through a band-pass filter 44 to select the wavelength range of interest for the excitation light. These filters are usually thin-film technology interference filters with on the order of 20 nm full-width-half-max (FWHM) bandpass and on the order of 60 to 90% peak transmission.

Another requirement of an illumination system is that the uniformity of light across the samples be uniform. If a large area is to be illuminated, as is the case of some implementations, uniformity is important. However, uniformity can be compromised, and compensated by correction factors. In some cases, this approach is advantageous.

Diffusers (not shown). One method for achieving this uniformity is to diffuse the light source. In one embodiment, holographic type diffusers are used to achieve high uniformity and efficiency. Both holographic and conventional diffusers are commonly available from optical suppliers. Of consideration here is that these types of diffusers will not maintain polarization and thus need to be used prior to the polarizer.

Lenses 42, 44. Lenses within the light-source assembly 40 serve to guide and direct light through the filters, diffusers, mirrors and other optical components in order to reach the sample.

Polarizer 46. The polarizer 46 used in the light-source assembly 40 can be fixed or variable, dependent on the approach selected, as described above. Simple polarizers are thin dichroic sheet material readily available in optics catalogs. More complex polarizers include Liquid-Crystal Polarizers (LCPs). These LCP devices can use the simple polarizers to first filter the incoming light to a linear state, and then are used to either passively let the linear light pass, or actively rotate the light via the Liquid-Crystal, to the opposing linear state.

Large crystal polarizers, such as a Glan-Thompson polarizer, can also be used. These polarizers are thick calcite crystal devices that they can efficiently deliver two polarizations simultaneously, but in different physical directions.

Retention of polarization is important in that the system must not significantly impact one polarization orientation over the other in an unpredictable fashion. Predetermine or fixed systematic biases can be measured and accounted for a-priori. These biases however, may be minimized so as not to significantly reduce the intensity of one of these signals over the other.

In some implementations, the light source module may include a fiber optic bundle to provide distinct sources of illumination for the individual sample wells. The fiber optic bundle can receive light from a single illumination source. In one example, these sources do not directly provide illumination to the well, but rather serve as a light source for an imaging system that projects light from the fibers to the wells, e.g., via a scanning mirror and other optics in the illumination path. In another example, each fiber directly illuminates a well, or a polarizing optical element designated for a discrete region of the sample carrier.

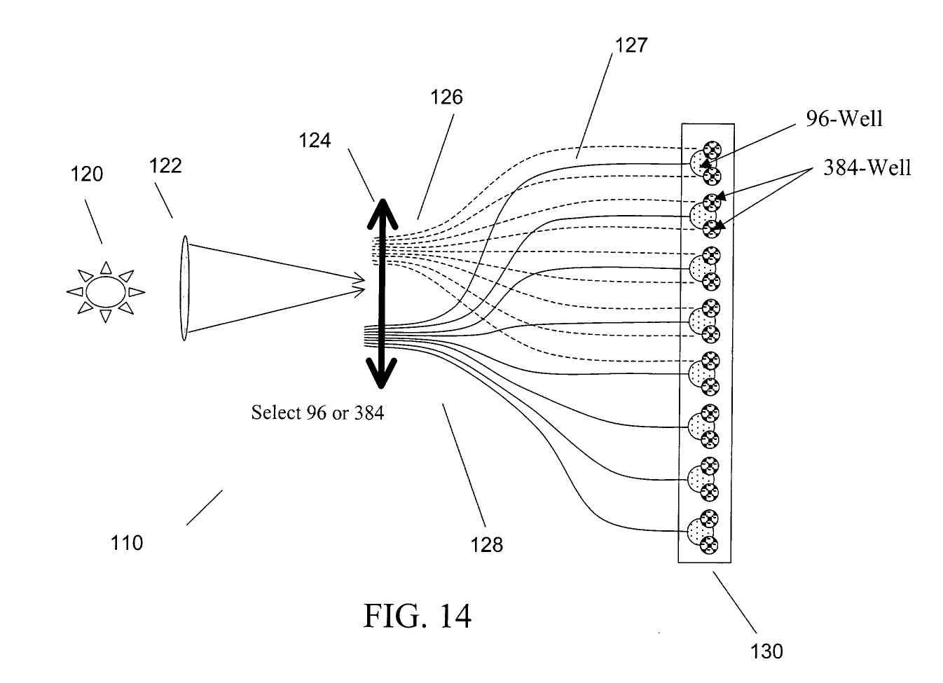

In another example, the fiber illumination system can illuminate either samples arranged using the separation spacing of a 96-well plate, or samples arranged using the separation spacing of a 384-well plate. Both fiber bundles are configured in an array, e.g., as shown in FIG. 14 (see also below). Then the fibers designated for the 96-well configuration are isolated into one bundle, and the 384-well configured fibers are isolated and directed into a second bundle. Since the fibers are flexible, the light source can remain stationary, and the appropriate bundle can be position to receive light from the light source. Equivalently, the fibers may remain stationary, and a mirror is moved to direct light to the appropriate fiber bundle. Typically, the fiber optic is not be utilized in the emission path, as that would perturb the spatial and polarization qualities of the image.

Referring to the example in FIG. 14, the system 110 includes a plurality of fiber bundles, e.g., two fiber bundles 126 and 128. One bundle is configured for a first configuration, e.g., a 96-well plate, and the other bundle is configured for a second configuration, e.g., a 384-well plate. As shown in FIG. 14, the fibers configured for the 96 well plate are located in bundle 128, and the 384-well configured fibers are isolated in bundle 126. In one embodiment, since the fibers may be flexible, the light source can remain stationary, and the appropriate bundle can be positioned to receive light from the source 120. For example, the appropriate bundle can be translated along the path 124 until it is in-line for illumination. In another embodiment, the fibers may remain stationary, and a mirror and/or lens system is moved to direct light to the appropriate fiber bundle.

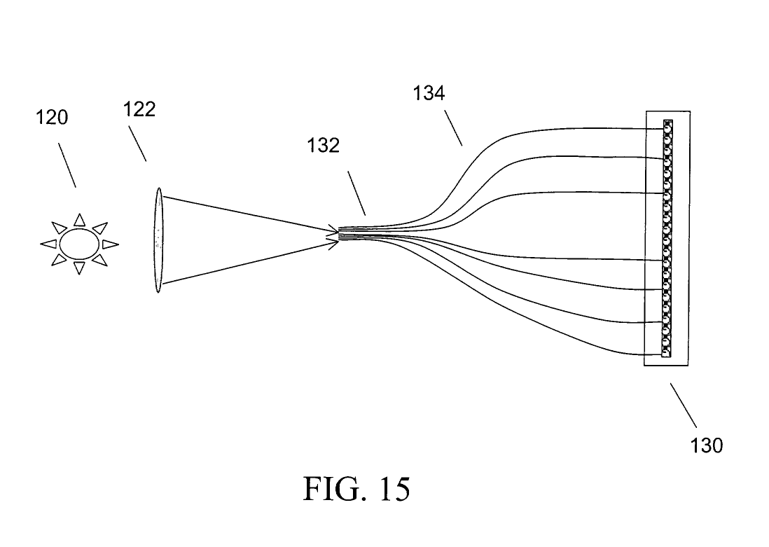

Referring to the related example in FIG. 15, a single bundle 132 of optical fibers 134 is illuminated by the light source 120. The individual fibers 134 are distributed to illuminate different regions of a sample carrier 130.

Detection Assembly

Referring again to FIG. 3, the detector 30 includes an imaging system 31, such as a single point detector or an array of detectors, commonly referred to as a camera. This detector or camera can be a single charged-coupled detector (CCD), an array of CCDs, a single photo-multiplier tube (PMT), or an array of PMTs. An intensifier 32 can be used to amplify the signal levels for those types of detectors where the inherent amplification is not sufficient. The detector 30 may have many of the same optical elements as the Light Source Assembly 40, depending on the particular configuration chosen.

Polarizer 36. Light emitted from the sample passes through a polarizer 36, which can be of the same construction as the polarizer 46 of the light source assembly. The polarizer 36 filters the light in the detection path such that only light of a particular polarization is detected. The relative orientation of the polarizers 36, 46 is important: Since polarization contrast is being measured, both the linear emission polarization parallel and perpendicular directions of polarization need to be measured. In another useful configuration, the excitation polarization direction can flip between the parallel and perpendicular orientations, whilst the emission polarizer stays in one orientation. In still another configuration, the emission polarization can be fixed in one direction and the emission polarizations can be separated into parallel and perpendicular components and measured independently and thus simultaneously. In addition, polarizers can be removed, e.g., to measure total fluorescence intensity.

Large crystal polarizers, such as a Glan-Thompson polarizer, can be used to analyze two different polarizations of emitted light in the detector 30.

Band-Pass Filter 34. The filters are emission filters that allow transmittance of light centered on the wavelength of the light emitted by the fluorophore. These filters are identical in function to that of the excitation filter, except that the center wavelength is shifted in wavelength according to the emission profile of the fluorophore.

The lenses are optimized for collecting light from the sample and delivering it through the filters in the detector 30 and to the camera 31.

Multiple pairs of excitation and emission filters (one of each make a pair) can be used for the various types of fluorophores that are used to monitor the PCR reaction. To assess multiple fluorophores in a single PCR reaction, the apparatus is outfitted with a plurality of these pairs.

The following are three exemplary configurations of an illumination system (e.g., light source assembly 40) and a detection system. Each of these configurations can be used to excite and/or detect multiple sites (e.g., multiple samples) at the same time or separately.

1. Sequential Detection. In this configuration, the illumination system is fixed such that polarized light illuminates the samples in a predetermined direction with an excitation beam of polarized light. The detection system is designed to sequentially analyze emitted light in at least two directions: perpendicular and parallel to the excitation beam.

For example, a polarizer in the detector 30 can be rotated 90° to switch between the detection of the two polarities (perpendicular and parallel). In another example, two polarizers are used, one for each polarity. In the detection path, the polarizer is removed from the light path and the other polarizer is inserted in order to switch polarities.

2. Sequential Excitation. In this configuration, the light source assembly 40 sequentially provides at least two beams of polarized light: one beam whose linear polarization is perpendicular to the direction of detected emitted light's polarization and the other parallel to the direction of detected emitted light's polarization. The detection system remains fixed and detects light emitted in a predetermined direction. As described for detector 30, the polarizer in the light source assembly 40 is rotated or switched in order to generate two different polarities of polarized light.

In one embodiment, both the sequential excitation and sequential detection are used. In an exemplary implementation of this embodiment, four measurements are made and averaged: two perpendicular detection measurements for each polarity of excitation light

3. Concurrent Detection. In this configuration, the illumination system is fixed such that polarized light illuminates the samples in a predetermined direction with an excitation beam of polarized light. The detection system simultaneously detects light emitted in two different polarization directions: one polarization direction perpendicular to the excitation beam and the other parallel to the polarization direction of the excitation beam. In one embodiment, the detection system includes two separate detectors that independently analyze emitted light in each respective detection path. In another embodiment, the beams of different polarization are split and recombined, but spatially separate on a single array type-detector that can isolate and identify the separate beams.

Concurrent detection of perpendicular and parallel polarized light can be implemented such that all samples are imaged at the same time or such that each sample is imaged individually.

One advantage of concurrent detection is speed. Since both readings are taken at the same time, additional time is not required to detect emitted light in the second direction. A second advantage is stability. The illumination for both directions of polarization is concurrent. Thus, measurements in the two directions result from the same amount of excitation light. Deviations in the illumination system that may result when the two measurements are made at two different points in time are avoided.

With respect to each of the three, above configurations, detection can be made such that an area that encompasses multiple samples on the sample carrier 23 are detected concurrently or such that individual samples are detected separately, e.g., sequentially.

In the scenario in which an area that encompasses multiple samples is detected concurrently, the area is illuminated by the light source assembly 40 and then detected using an imaging system, e.g., a system that includes an array that assigns values to different pixels of an image. This scenario has, among others, the advantage of speed.

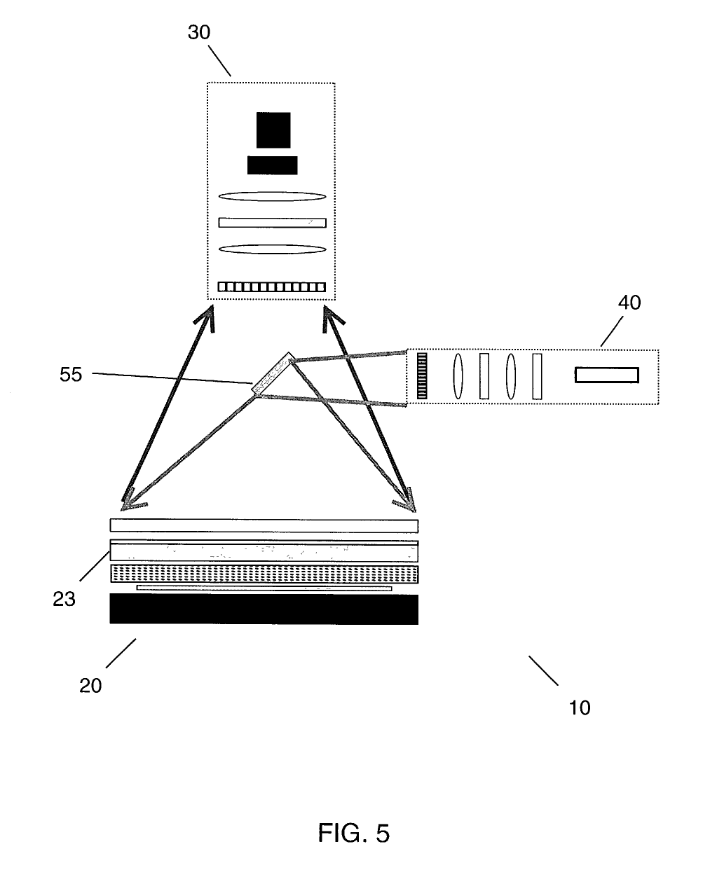

In the scenario in which individual samples are detected separately, a scanning system is used to selectively illuminate and/or selectively detect emitted light from a particular individual sample. In a preferred embodiment, the sample carrier 23 is fixed throughout the process. However, the optics are modified to scan the different individual samples. For example, the scanning system can include a moveable mirror, e.g., the scanning mirror 55 of FIG. 5.

The scanning system has the advantage that the illumination can be directed at each sample individually or at a subset of samples, potentially requiring less total illumination and less interference from parts of the system that would be illuminated if the whole sample carrier 23 was illuminated. The scanning system can use a single point detector, such as a PMT, which is very sensitive and has a great dynamic range, or an array of point detectors. Additionally, by illuminating a single or small number of samples at a time, the amount of illumination per sample can be significantly increased, while avoiding photobleaching of areas not illuminated and not being detected.

In addition to the variety of configurations above, the detection system and light source can be configured for transient-state or steady-state detection. For steady-state detection, the excitation light is provided during the interval during which the detection system detects emitted light. In contrast, for transient-state detection, the excitation light is provided at one time. A temporal delay follows, after which the detection system detects emitted light. In this configuration, the detection system does not receive noise in the form of reflected excitation light that pass through the bandpass filters on the detection path.

The following are some exemplary apparati 10.

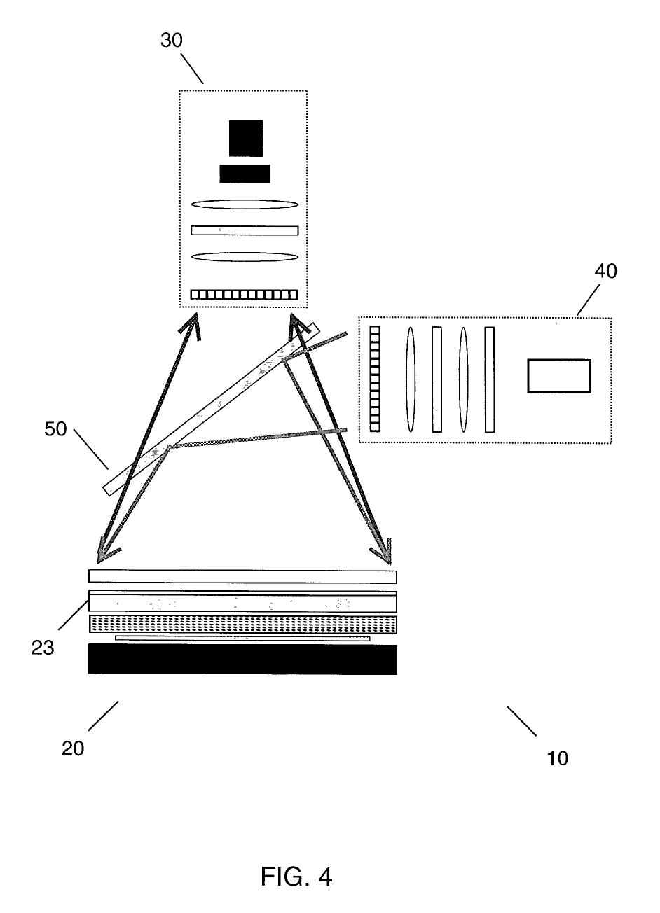

Referring to the example in FIG. 4, the light source assembly 40 produces a beam of excitation light that is parallel to the surface of the sample carrier 23. The beam is reflected by a beam splitter 50 to direct the beam onto the surface of the sample carrier 23. The beam illuminates a sufficient area of the surface such that multiple different samples within the sample carrier receive the excitation light. Fluorescent light emitted by samples in the sample carrier then travels to detector 30, passing through the beam splitter 50.

Referring to the example in FIG. 5, the light source assembly 40 produces a beam of excitation light that is parallel to the surface of the sample carrier 23. The beam is reflected by a scanning spot mirror 55 that direct the beam onto the surface of the sample carrier. The beam illuminates a sufficient area of the surface such that multiple different samples within the sample carrier receive the excitation light. The mirror can be coupled to a control unit that positions the mirror in order to illuminate specific areas on the surface of the sample carrier. Fluorescent light emitted by samples in the sample carrier then travels to detector 30.

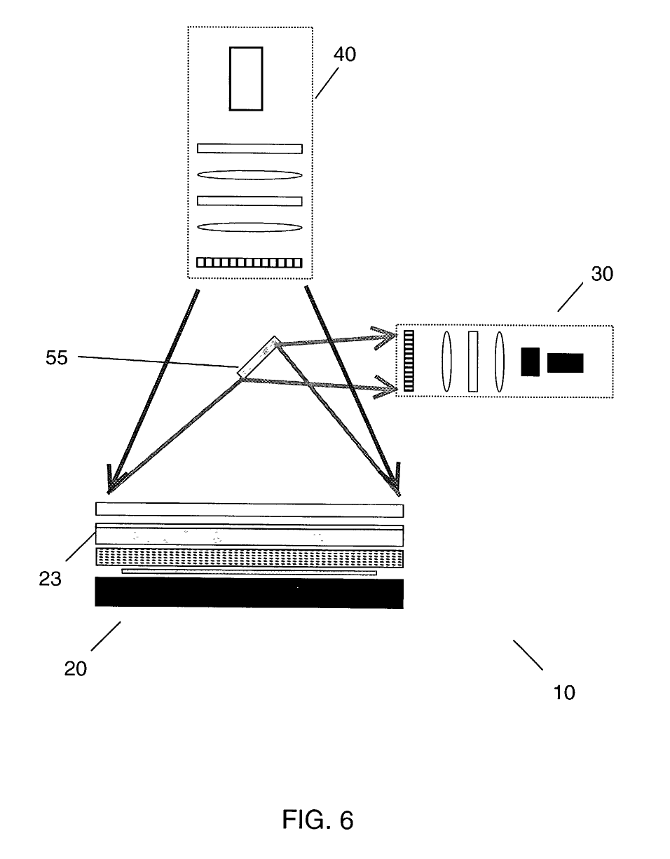

Referring to the example in FIG. 6, the light source assembly 40 can be positioned facing the sample carrier 23. In this configuration, the detector 30 is now positioned to receive emitted light along a path parallel to the surface of the sample carrier. The scanning spot mirror 55 directs emitted light from areas on the surface of the sample carrier 23 into the detector 30. The configuration in FIG. 6 resembles that of FIG. 5, except the location of the light source assembly 40 and the detector 30 are switched.

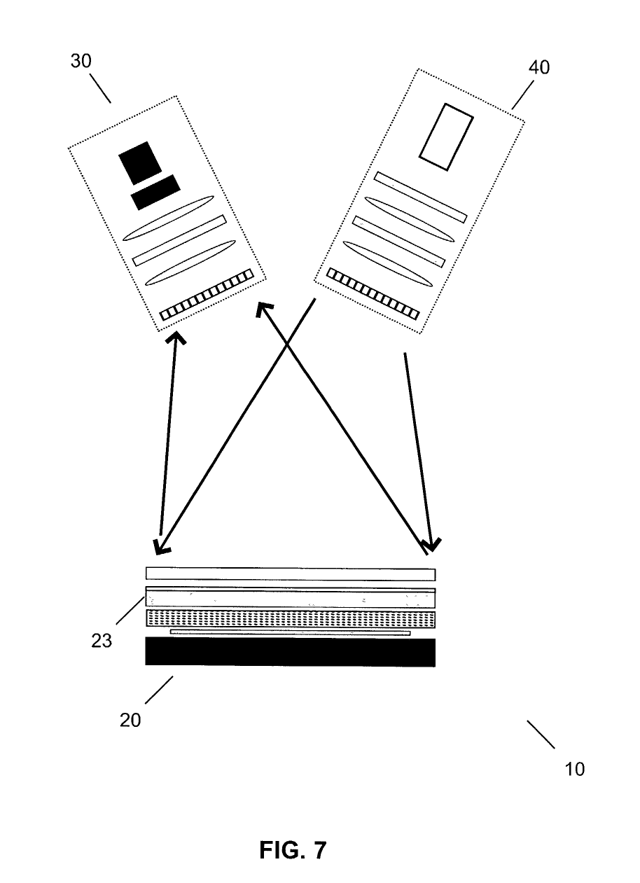

Referring to the example in FIG. 7, both the light source assembly 40 and the detector 30 are positioned facing the area on the surface of the sample carrier 23 to be detected. As shown in FIG. 7, the path from the light source assembly 40 and the surface and the path from the surface to the detector 30 are both oblique. In a related embodiment, the light source assembly 40 or the detector 30 is located such that the light path is perpendicular to the surface of the sample carrier 23. However, the unit that is not so located is positioned such that the path between it and the surface is oblique.

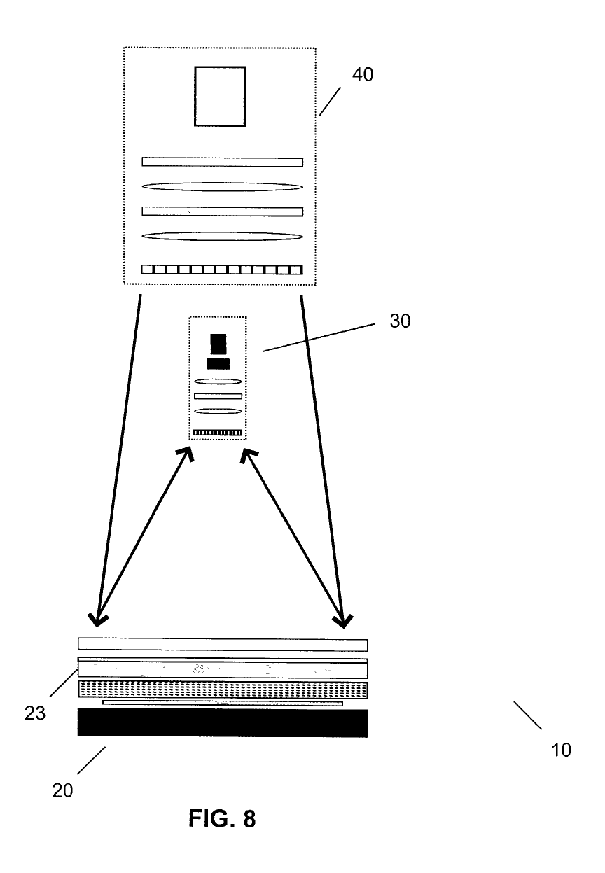

Referring to the example in FIG. 8, the detector 30 is located within the path of the excitation light from the light source assembly 40 and the sample carrier 23. For example, the detector 30 is position to only block a small region of the area illuminated by the beam of excitation light.

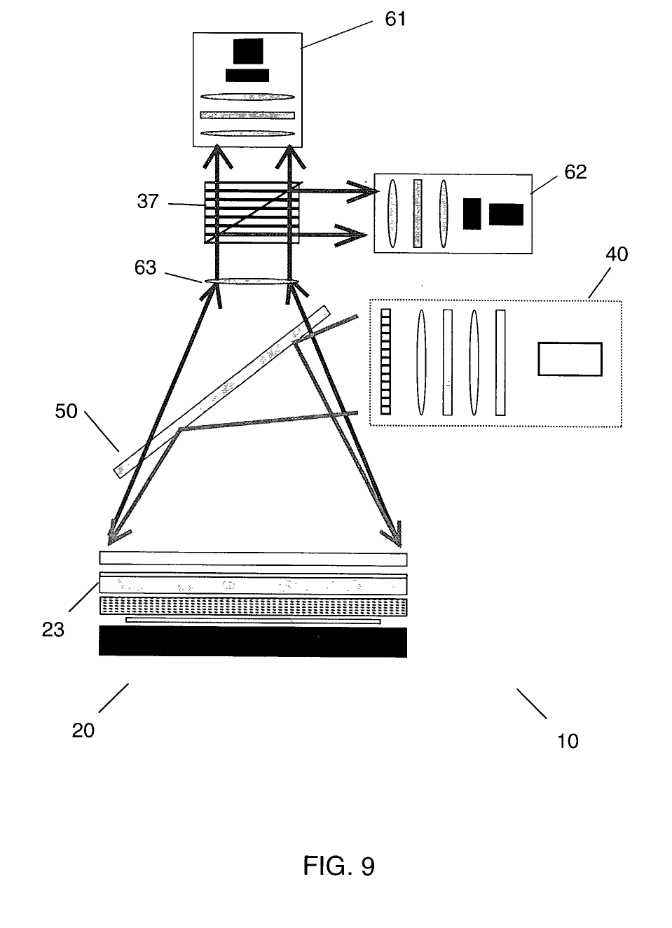

Referring to the example in FIG. 9, two detectors 61, 62 are used with a beam splitting polarizer 37. Emissions light is collected with a lens 63 and then directed through the beam splitting polarizer 37 (e.g., a Glan-Thompson polarizer, thin-film beamsplitter, or microwire type beamsplitter). Light polarized in one direction travels to the first detector 61. Light polarized in a perpendicular plane travels to the second detector 62. This configuration enables concurrent detection of light polarized parallel to the excitation beam and light polarized perpendicular to the excitation beam. See, the “Concurrent Detection” configuration described above.

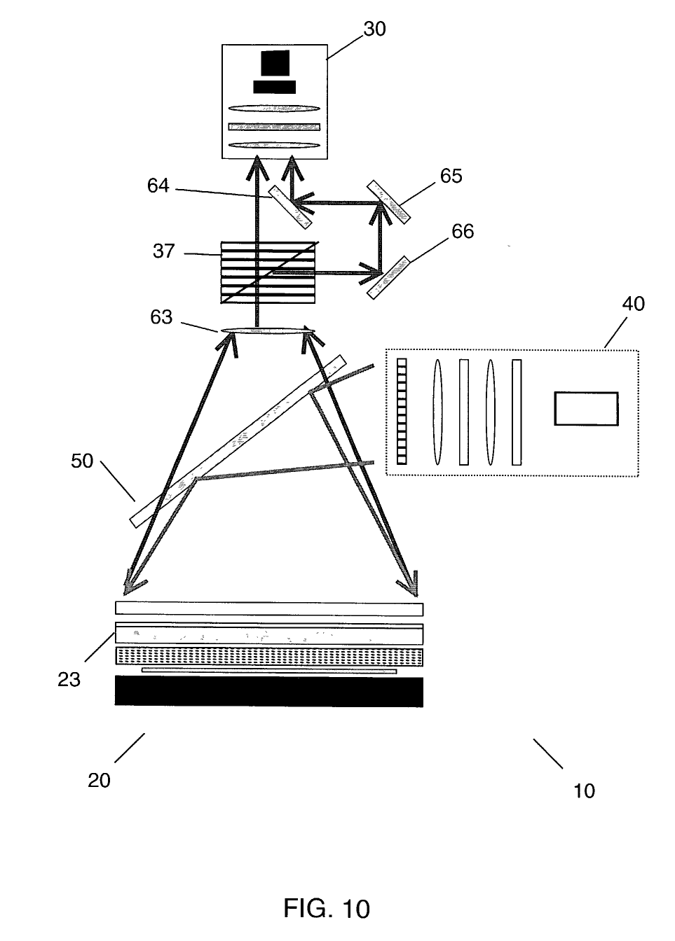

Referring to the example in FIG. 10, one detector 30 is used for concurrent detection of light polarized parallel to the excitation beam and light polarized perpendicular to the excitation beam. Emissions light is again collected with a lens 63 and then directed through the beam splitting polarizer 37. Light polarized in one direction travels directly to the detector 30. Light polarized in a perpendicular plane is reflected by mirrors 64, 65, 66 in to the detector 30. Hence, the detector 30 reads two images of the sample carrier 23, one image for each polarity of light.

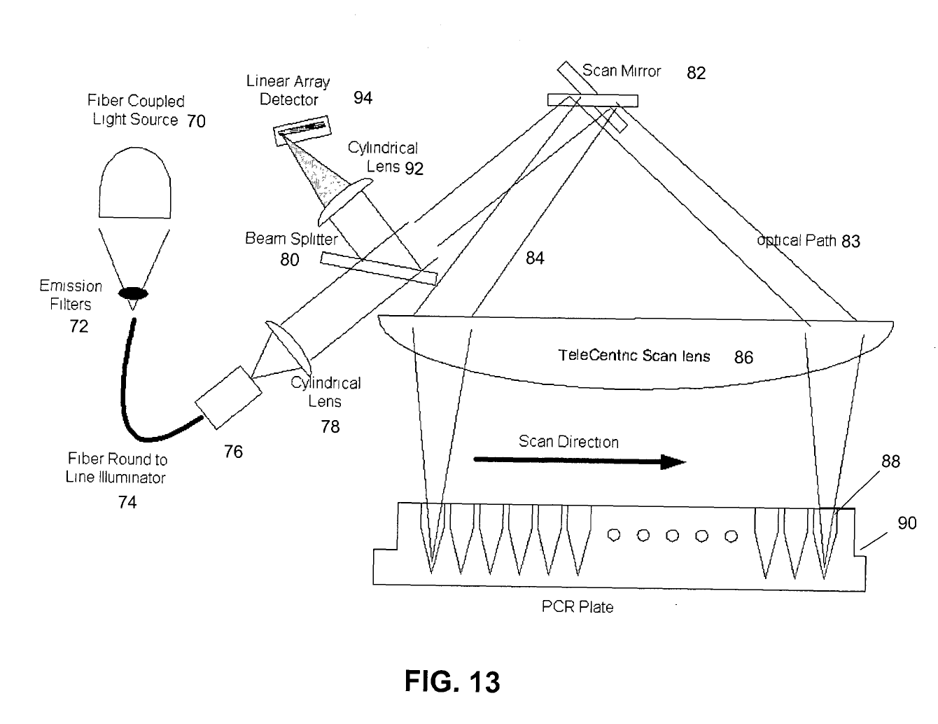

Referring to the example in FIG. 13, excitation light is provided by a fiber-coupled light source 70. The light is filtered to a desired excitation wavelength by filters 72. A fiber 74 channels the light to the line illuminator 76. Typically, the light is polarized subsequent to the fiber optic by the polarizer and prior to reaching the scanning mirror 82, although light can be polarized prior to the fiber if the fibers preserve the state of polarization. The light beam is focused by a cylindrical lens 78 and directed by a scanning mirror 82 to a region of the sample carrier 90. Two possible optical paths 83, 84 are shown. These paths pass through a telecentric scan lens 86 which focuses the beam, for example, on a region 88 of the sample carrier 90. The region may be, for example, one well of a microtitre plate, more typically a row of wells, or more generally any area that may include a plurality of different nucleic acid samples. Light emitted by a fluorophore within the area travels back to the scanning mirror 82 and is reflected by the beam splitter 80 to a cylindrical lens 92 which focuses the light onto a linear array detector 94. In a related implementation, the telecentric scan lens is replaced by an array of lenses.

System for FP Monitoring

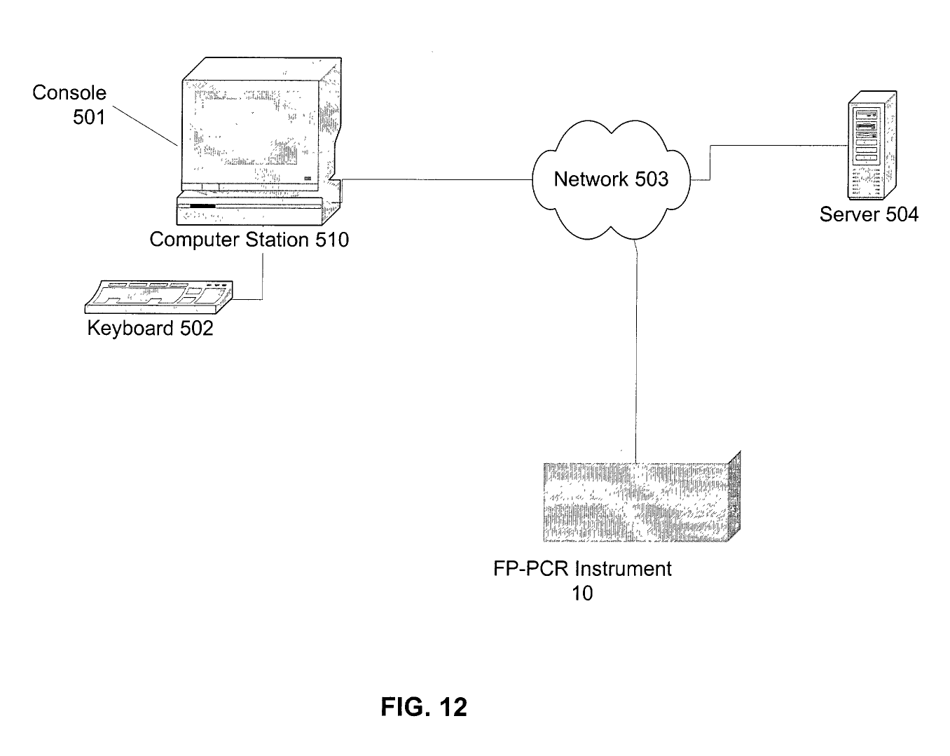

Also featured is a system for FP monitoring. Referring to the example in FIG. 12, the system includes: a computer system 510 that is in signal communication with the FP-PCR apparatus 10. The computer system 510 includes a console 501, keyboard 502, and so forth. The computer system 510 can be connected to a network 503 which includes a server 504.

For example, the computer system 510 can interface with a user to customize an FP-PCR procedure. The computer system 510 can send instructions to the apparatus 10 in order to execute the procedure. The instructions, for example, directly control the thermal cycler assembly 20 and optical assembly 15 to execute the procedure.

In another implementation, the computer system 510 sends the user-entered parameters to the apparatus 10. A processor on-board the apparatus 10 then controls the thermal cycler assembly 20 and optical assembly 15 to execute the procedure.

The computer system 510 also receives information from the detector 30. The information can be unprocessed images obtained by the detector 30, or images preprocessed by the detector 30. Software executed by the computer system 510 can be used to process the images and obtain readings for each sample in the sample carrier 23. The readings, typically obtained in real-time, are stored. Information can also be displayed on the console 501 during the PCR procedure, e.g., to provide preliminary results to the user. After the PCR procedure is completed, the stored readings are completely processed, e.g., to determine the initial concentration of particular nucleic acid species in the initial sample. One algorithm for determining the initial concentration is described below (see, “Real-Time Amplification Algorithm”)

Similar systems can be used to monitor other nucleic acid amplification reactions, e.g., isothermal reactions.

Real-Time Amplification Algorithm

In one aspect, the invention features a method for determining target nucleic acid concentration for a sample (and for a plurality of samples, e.g., in a plurality of reactions, e.g., in each well of a multi-well plate, at one or more locations on a substrate, in one or more capillary tubes, etc). The method can use one or more reference samples to compare and then extrapolate observed values to an initial starting nucleic acid concentration for a sample. The observed values are directly proportional to the concentration of nucleic acid. The observed values, however, can be any measure that indicates nucleic acid concentration, e.g., an FP value in the example of fluorescence polarization, or an fluorescence intensity (FI) value, e.g., in the case of a 5′ nuclease probe whose fluorescence intensity changes with nucleic acid concentration. FP measurements are related to Fl by equation 1:

FI=Fₚₐᵣₐₗₗₑₗ+2*F_{perpendicular} (1)

where Fₚₐᵣₐₗₗₑₗ is the fluorescence signal of light polarized in the plane of the excitation light, and F_{perpendicular} is the fluorescence signal of light polarized in the plane perpendicular to the excitation light.

The following example, describes the relationship between F and an absolute measurement of concentration. F is any value that is directly proportional to concentration, e.g., an FP value and or an FI value (e.g., of a nuclease assay), depending on the implementation. F values are converted to an absolute measurement of concentration as follows. Concentration is directly proportional to fluorescence. This means that for a given percentage change in fluorescence, the change in concentration is of an equal percentage.

For a typical reaction, the F signal is flat until the DNA is amplified a sufficient enough times such that the sample signal is above the noise of the system. (e.g., instrument and sample noise). Once the signal is above the system noise by some multiple of the noise level, the curve becomes exponential, such that the logarithm of the signal (ln(Fₜ)) is linear with respect to time (e.g., PCR cycle number). As the sample reaches saturation, this relationship (ln(Fₜ) vs. time) is no longer linear.

The linear section of the curve is identified and then used to linearly extrapolate to the vertical axis at time-equals-zero (t₀) to determine the value of ln(F₀), and thus F₀, by taking the inverse logarithm. In this example, the initial fluorescence (KF₀) (i.e., at to) is inferred from the intercept ln(FP₀) of the y-axis (the ln(FP) axis) by raising e to the ln(FP₀) power, i.e., KF₀=FP₀.

The initial concentration of target nucleic acid is then determined from F₀, e.g., by use of a reference to a standard of known concentration. For example, the initial F₀ values can be determined for a sample of unknown concentration (UC₀) and a known concentration (KC₀) on the same instrument in the same run, e.g., using the method described in the previous paragraph. For a known concentration sample and an unknown concentration of sample, the conversion can be easily performed. The calculated to fluorescence of the known concentration sample (KF₀) becomes the standard reference, and the calculated to fluorescence of the unknown concentration sample (UC₀) is the unknown desired data.

$$\frac{{KC}_{0}}{{KF}_{0}} = \frac{{UC}_{0}}{{UF}_{0}} \qquad ( 2 )$$

Equation 2 is rearranged as follows:

$${UC}_{0} = \frac{{KC}_{0}}{{KF}_{0}} {UF}_{0} \qquad ( 3 )$$

This is clear once the relationship between fluorescence and concentration is shown explicitly, and the fact that the relationship between the concentration and the fluorescence of the known and unknown samples have to be the same. The following equation is true for this relationship.

KF=α·KC and UF=α·UC, (4)

where α is the proportionality constant. Then using equation (4) in equation (3), dropping the subscripts, it becomes clear that the equation is valid,

$$UC = \frac{KC}{KF} UF = \frac{KC}{( α \cdot KC )} \cdot ( α \cdot UC ) = UC \qquad ( 5 )$$

An example of this is as follows. If a sample of known concentration KC₀=10,000 is placed in the device and the to fluorescence (KF₀) is calculated to be 20,000; and an unknown concentration of sample is placed in the device and the to fluorescence of the unknown (UF₀) is calculated to be 50,000, then the equation will yield a concentration (UC₀) of 25,000 for the unknown.

$${UC}_{0} = \frac{10 , 000}{20 , 000} 50 , 000 = 25 , 000 \qquad ( 6 )$$

Here, for the reference sample (Known), the fluorescence is twice as much as the concentration, so for the unknown, the concentration must be half that of the fluorescence, with the relationship being implicit, and alpha being two.

These calculations can be automatically determined by software. The software can be linked to the apparatus to automatically receive and process data. The software can include a user interface to receive user instructions and to query the user, e.g., to determine sample identities, concentrations of known control samples, report formats, and so on.

In another implementation, the software is operated independently of the apparatus, e.g., on a desktop computer or handheld device. For example, data from the instrument can be manually loaded (or entered) for analysis.

In some implementations, the above algorithm can be used without assaying a known dilution series of DNA as a calibration tool. Further, since the algorithm is independent of the slope of the ln(FPₜ) vs. time curve, accurate results are generate even if different samples have different efficiencies of amplification.

The above algorithm may be applicable to any method for real-time monitoring nucleic acid amplification, e.g., methods other than FP monitoring.

Amplification Reactions

Biochemical procedures for PCR amplification are generally described, for example, in: Dieffenbach and Dveksler (1995) PCR Primer: A Laboratory Manual, Cold Spring Harbor Laboratory Press; Sambrook & Russell (2001) Molecular Cloning: A Laboratory Manual, 3^{rd} Edition, Cold Spring Harbor Laboratory Press; U.S. Pat. Nos. 4,683,195 and 4,683,202, Saiki, et al. (1985) Science 230, 1350-1354.

A typical FP-PCR amplification reaction includes the following components:

thermostable DNA polymerase

deoxynucleotides

a forward primer

a reverse primer

buffer and salts (e.g., 10 mM KCl, 10 mM (NH₄)2SO_{4, 20} mM Tris-HCl, 2 mM MgSO₄, 0.1% Triton X-100, pH 8.8)

The forward and reverse primers are designed to specifically anneal to respective ends of a target sequence that is to be detected. For FP-PCR, one of the two primers of the pair is labeled with a fluorophore.

Exemplary fluorophores for FP-PCR include: fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7.

In one implementation, a mixture is prepared with the amplification reaction components. Aliquots of the mixture are distributed into different wells in the microtitre plate sample carrier. Different samples are added to each of the wells. If desired, some of the wells can be used to prepare a dilution series for one or more of the samples. However, in some embodiments, accurate FP detection and appropriate algorithmic usage obviates the need for a dilution series to a quantitative measure of the initial target sequence concentration in various samples.

Temperature cycling: For FP-PCR, a standard PCR cycle can be used. For example, cycling between a denaturing temperature, an annealing temperature, and a primer extension temperature. Particular temperatures and times can depend on particular implementation details, e.g., on primer design, primer binding site sequence, and length of the amplified target sequence length.

As mentioned herein, in one embodiment, the heat-transfer block 24 provides a thermal gradient. Thus, annealing temperatures, for example, can be varied among wells of a sample carrier 23.

Measurment of FP. FP is affected by temperature, among other factors. Hence, data is acquired from the sample carrier at a particular temperature during the thermal cycle. For example, one convenient temperature is between 40 and 70° C., 55-65, 37-42, or 65-75° C. The temperature can be a temperature at which unextended primers are annealed to binding sites on their complement (if present) or a temperature at which unextended primers are not annealed to their complements.

The PCR cycle can also be programmed to hold the sample carrier temperature at a temperature suitable for data acquisition once every cycle. In some implementations, a thermal probe is attached to the sample carrier. The probe can be inserted directly into the solution in one of the wells of the carrier. Temperature readings from the probe are used to trigger FP data acquisition. A record of the temperature can also be stored.

Linear PCR. In one embodiment, the PCR amplification is linear with respect to concentration of extended primers and time. Only a single primer is used for linear PCR. In other words, a reverse primer is not used. Amplification proceeds linearly with time since during each cycle the number of extended primers formed is equal to the number of target molecules present in the initial sample. The slope of the plot of extended primer concentration vs. time can be used to determine the number of initial molecules. Linear PCR, therefore, can be used to obtain very accurate measures of target molecule concentrations in the initial sample, provided the amount is sufficient for detection by linear amplification.

The methods and apparati can also be adapted to other nucleic acid amplification techniques. Some other examples include:

transcription-based methods that utilize, for example, RNA synthesis by RNA polymerases to amplify nucleic acid (U.S. Pat. No 6,066,457; U.S. Pat. No 6,132,997; U.S. Pat. No 5,716,785; Sarkar et. al., Science (1989) 244: 331-34; Stofler et al., Science (1988) 239: 491; U.S. Pat. Nos. 5,130,238; 5,409,818; and 5,554,517 (for NASBA) and strand displacement amplification (SDA; U.S. Pat. Nos. 5,455,166 and 5,624,825);

ligase chain reaction (LCR). With respect to LCR, since the ligation of a labeled probe to a small unlabeled oligonucleotide may only result in a small difference in FP, the labeled probe can be ligated to a large, unlabeled molecule in order to increase the change in FP signal upon ligation; and

a flap endonuclease-based cleavage, e.g., as described in U.S. Pat. No. 5,888,70 and 6,001,567.

With respect to some of these other amplification techniques, amplification can be isothermal. The light assembly 20 can sample the reaction mixture (or mixtures) at multiple intervals during the amplification. Typically, regular intervals are chosen.

Multiplex Primer Analysis

More than one target nucleic acid sequence can be analyzed at one or more discrete addresses of a reaction chamber (e.g., samples of a sample carrier, e.g., wells of a microtitre plate). A different labeled primer is used for each target sequence. For example, two primers that amplify related or unrelated sequences are labeled with different fluorophores.

To detect two alleles of a gene, the reaction can include

a first primer specific for the first allele and labeled with a first fluorophore;

a second primer specific for the second allele and labeled with a second fluorophore; and

a third primer that binds to both the first and second allele, on the apposing strand.

If the first allele is present, the first and third primer amplify the target sequence. If the second allele is present, the second and third primer amplify the target sequence. If the allele is an SNP, the inappropriate primer may hybridize and prime synthesis of the allele that is present. However, quantitative detection would, nevertheless, indicate preferential amplification by the appropriate primer. In addition, the primers' query position which distinguish the SNP may be judiciously positioned, e.g., at or near the 3′ terminus of the primer (e.g., within 1, 2, 3, 4 or 5 nucleotides of the terminus). The primer can also include deliberate mismatches, e.g., adjacent to or near the query position, to decrease the Tm of the primer and increase its sensitivity.

To detect two unrelated target nucleic acids, the reaction can include:

a first primer specific for the first nucleic acid and labeled with a first fluorophore;

a second primer specific for the first nucleic acid, and hybridizing to a site on the first nucleic acid such that a segment of the nucleic acid is amplified in combination with the first primer.

a third primer specific for the second nucleic acid and labeled with a second fluorophore; and

a fourth primer specific for the second nucleic acid and hybridizing to a site on the second nucleic acid, such that a segment of the nucleic acid is amplified in combination with the third primer.

The two unrelated nucleic acids might be genes transcribed by the same cell, e.g., genes encoding actin and p53. In another example, the two unrelated genes might be an antibiotic resistance gene and a gene indicative of bacterial virulence.

Multiple different fluorophores (e.g., at least two, three, four, five, or six different fluorophores) can be used in a multiplex analysis. An exemplary set of six includes: (1) 6-FAM; (2) HEX; (3) Texas Red; (4) Cy5; (5) Cy5.5; and (6) a fluorophore selected from the following group: Cy3, Pacific Blue, TAMRA, and Cy7. In general, any set of fluorophores for which the emission and/or excitation peaks are separable can be used. Moreover, both need not be separable, so long as they can be separated by detection or by excitation.

Intercalating Dyes

It is also possible to use an intercalating dye in an implementation that does not require the amplification primer to be fluorescently labeled (although it may be with a different dye that does not interfere). Sybr Green is an intercalating dye that binds to the minor grooves in double-stranded DNA. Ethidium bromide is another example. The dye is relatively inactive when unbound in solution, but becomes much brighter when bound to DNA. Thus, as the amount of DNA increases during a PCR reaction, the signal of Sybr Green increases in proportion, as the dye binds to each new PCR product as can be detected by a standard real-time PCR instrument using a top-read prompt fluorescence mode. The unbound dye, however, emits a signal, which is detectable above background although weak compared to the bound dye.

Testing has demonstrated that Sybr Green monitoring in FP mode in real-time amplification provides useful information for quantitating amplification. The signal of the unbound dye is pronounced enough to detect the contrast needed to see an increase in mP value as more dye binds to DNA over time. The result is a large change (increase) in mP value between the 1^{st} PCR cycle and the last (i.e. ₃₅-40^{th}) cycle.

Thus, the FP curve of intercalating dyes (including Sybr Green) are indicators of the extent of product formation in any nucleic acid reaction, including nucleic acid amplification reactions, such as PCR or isothermal amplification reactions that produce a double-stranded product.

Software

The computer-based aspects of the invention can be implemented in digital electronic circuitry, or in computer hardware, firmware, software, or in combinations thereof. Algorithms and control procedures described herein can be implemented in a computer program product tangibly embodied in a machine-readable storage device for execution by a programmable processor; and method actions can be performed by a programmable processor executing a program of instructions to perform functions of the invention by operating on input data and generating output. Output can include information for a user (e.g., graphics or values, e.g., representing inferred nucleic acid concentrations) and/or commands for controlling an FP apparatus. Input can include receiving signals, e.g., signals representing information from detected light of an FP apparatus.

The invention can be implemented advantageously in one or more computer programs that are executable on a programmable system including at least one programmable processor coupled to receive data and instructions from, and to transmit data and instructions to, a data storage system, at least one input device, and at least one output device (e.g., a printer, console, FP apparatus, or disc drive). Each computer program can be implemented in a high-level procedural or object oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks.

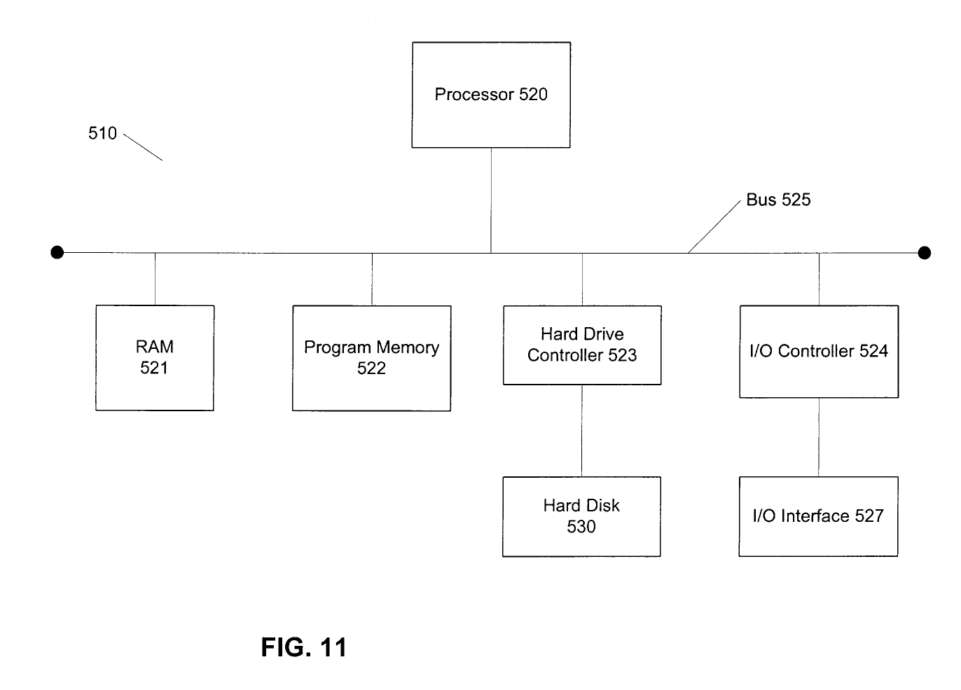

An example of one such type of computer is shown in FIG. 11, which shows a block diagram of a programmable processing system 510 suitable for implementing or performing the apparatus or methods of the invention. The computer system 510 includes a processor 520, a random access memory (RAM) 521, a program memory 522 (for example, a writable read-only memory (ROM) such as a flash ROM), a hard drive controller 523, and an input/output (I/O) controller 524 coupled by a processor (CPU) bus 525. The computer system 510 can be preprogrammed, in ROM, for example, or it can be programmed (and reprogrammed) by loading a program from another source (for example, from a floppy disk, a CD-ROM, or another computer).

The hard drive controller 523 is coupled to a hard disk 530 suitable for storing executable computer programs, including programs embodying the present invention, and data including storage. The I/O controller 524 is coupled by means of an I/O bus 526 to an I/O interface 527. The I/O interface 527 receives and transmits data in analog or digital form over communication links such as a serial link, local area network, wireless link, and parallel link.

One non-limiting example of an execution environment includes computers running Windows NT 4.0 (Microsoft) or better or Solaris 2.6 or better (Sun Microsystems) operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. Alternatively, a Solaris 2.6 Ultra 10 (400 Mhz) with 256 MB memory and 9 GB SCSI drive can be used.

Exemplary Applications

FP detection of amplification reactions can be used broadly, e.g., to quantitate the abundance of particular nucleic acids. Exemplary applications include: detecting levels of gene expression in a sample, detecting the presence of an oncogene in a sample, detecting the presence of a cancer cell in a sample, detecting a single-nucleotide polymorphism in a sample (e.g., a blood sample or forensic sample), detecting a pathogen in a sample, genotyping a sample (e.g., for diagnostics or forensics), and RNA splice detection.

Other Embodiments

A number of embodiments of the invention have been described. Nevertheless, it will be understood that various modifications may be made without departing from the spirit and scope of the invention. Accordingly, other embodiments are within the scope of the following claims.

What is claimed: Apparatus

1. An apparatus comprising:
a sample carrier that comprises spatially distinct nucleic acid samples;
a light source configured to concurrently excite fluorescent compounds, located in a plurality of spatially distinguishable areas within a first region of the sample carrier, with polarized light;
a detection system configured to concurrently detect emitted light from the fluorescent compounds in each of the areas of the plurality in the first region; and
a thermal control unit configured to regulate the temperature of the sample carrier.

2. The apparatus of claim 1 wherein the thermal control unit is configured to cyclically heat and cool the carrier during a reaction course.

3. The apparatus of claim 1 wherein the sample carrier is immobilized relative to one or both of the detection system and the light source.

4. The apparatus of claim 1 wherein the sample carrier comprises a plurality of physically bounded areas.

5. The apparatus of claim 4 wherein each area of the plurality comprises a container within the sample carrier.

6. The apparatus of claim 1 wherein the detection system is further configured to detect emitted light of a first polarity and emitted light of a second polarity.

7. The apparatus of claim 6 wherein the first and second polarities are approximately orthogonal to each other.

8. The apparatus of claim 6 wherein the first polarity is parallel to the polarity of the polarized light from the light source, and the second polarity is non-parallel to the polarity of the polarized light from the light source.

9. The apparatus of claim 6 wherein the detection system is configured to detect the first and second polarity light concurrently.

10. The apparatus of claim 6 wherein the detection system comprises a first and second detector.

11. The apparatus of claim 9 wherein the detection system has a single detector, and the first and second polarity light are projected onto different regions of the detector.

12. The apparatus of claim 6 wherein the detection system is further configured to detect the first and second polarity light sequentially.

13. The apparatus of claim 12 wherein the detection system comprises a polarizer that is controlled to enable sequential detection of the first and second polarity light.

14. The apparatus of claim 1 wherein the detection system is further configured to distinguish polarized light from a first fluorophore from polarized light from a second fluorophore.

15. The apparatus of claim 6 wherein the first region comprises all physically distinct samples of the sample carrier.

16. The apparatus of claim 1 wherein the apparatus further comprises a scanning mirror.

17. The apparatus of claim 16 wherein the scanning mirror reflects excitation light from a light source.

18. The apparatus of claim 16 wherein the scanning mirror reflects emitted light from the sample carrier to a detector.

19. The apparatus of claim 1 wherein the light path emanating from the light source is parallel to the incident light path into the detector.

20. The apparatus of claim 19 wherein the detector is positioned between the light source and an imageable surface of the sample carrier.

21. The apparatus of claim 1 wherein the light path from the light source to the sample carrier surface or the light path from an imageable surface of the sample carrier to the detector is oblique with respect to the imageable surface.

22. The apparatus of claim 1 wherein the light source is further configured to excite a second region and the detection system is further configured to detect emitted light from the second region.

23. The apparatus of claim 1 wherein the detection system is configured to detect light in a plane parallel to the polarized light from the light source.

24. The-apparatus of claim 1 wherein the detection system and light source are in signal communication to enable a temporal delay between excitation and detection.

25. An apparatus comprising:
a plurality of spatially distinguishable reaction samples, each comprising amplification reagents that include a nucleic acid primer that is attached to a fluorophore;
an amplification control unit that is configured to control conditions of the reaction samples for nucleic acid amplification;
a fluorescence polarization monitor that is configured to concurrently monitor fluorescence polarization associated with each reaction sample of the plurality.

26. The apparatus of claim 25 wherein the fluorescence polarization monitor comprises a source of polarized light and a detector that can concurrently monitor emitted light from each sample of the plurality of reaction samples.

27. The apparatus of claim 26 wherein the detector is configured to concurrently monitor emitted light of a predetermined polarity.

28. The apparatus of claim 27 wherein the detector is configured to sequentially detect light of a first polarity and light of a second polarity, the light of the first polarity being parallel to the plane of the polarized light from the source.

29. The apparatus of claim 28 wherein the first and second polarities are orthogonal to each other.

30. The apparatus of claim 25 wherein at least some of the samples comprise a second nucleic acid primer that is attached to a second fluorophore that is spectrally distinguishable from the first fluorophore, and the detector comprises optical filters that can distinguish emitted light from the first and second fluorophore.

31. A method comprising:
providing a plurality of spatially distinct nucleic acid samples and amplification reagents that comprises a fluorophore attached to a nucleic acid primer;
concurrently amplifying each sample of the plurality; and
during the amplifying, concurrently detecting fluorescence polarization information associated with-the fluorophore from each sample of the plurality.

32. The method of claim 31 wherein the detecting comprises detecting fluorescence polarization information at at least a plurality of instances during the amplifying.

33. The method of claim 31 wherein the amplifying comprises thermal cycles and the detecting comprises detecting fluorescence polarization information at at least one instance for each cycle.

34. The method of claim 33 wherein the at least one instance for each cycle is at a predetermined temperature of the cycle.

35. The method of claim 31 wherein the amplifying and detecting are effected by an apparatus comprising
a light source configured to concurrently excite the fluorophores, located in a plurality of the spatially distinct samples, with polarized light; and
a detection system configured to concurrently detect emitted light from the fluorophores in each of the spatially distinct samples of the plurality.

36. The method of claim 31 wherein the amplifying comprises PCR amplification.

37. The method of claim 36 wherein the PCR amplification comprises exponential amplification.

38. The method of claim 36 wherein the PCR amplification comprises linear amplification.

39. The method of claim 31 wherein the detecting comprises exciting the fluorophore with polarized excitation light and detecting emitted light in a first predetermined plane.

40. The method of claim 39 wherein the first predetermined plane is parallel to the plane of the polarized excitation light.

41. The method of claim 40 wherein the detecting further comprises detecting emitted light in a second predetermined plane.

42. The method of claim 41 wherein the emitted light in the first and second predetermined planes are detected concurrently.

43. The method of claim 41 wherein the emitted light in the first and second predetermined planes are detected at separate times.

44. The method of claim 35 wherein each of the samples is disposed in a separate address of a sample carrier.

45. The method of claim 44 wherein the sample carrier is stationary relative to the light source and/or detection system throughout the amplifying.

46. A method comprising:
providing a reaction mixture that include a nucleic acid sample, amplification reagents, and a fluorescent probe that is bindable to double-stranded nucleic acid and has at least a 10-fold preference for double-stranded nucleic acid relative to single-stranded nucleic acid;
amplifying each sample of the plurality; and
during the amplifying, detecting fluorescence polarization information associated with the fluorescent probe at at least a plurality of instances.

47. The method of claim 46 wherein the fluorescent probe is an intercalating dye.

48. The method of claim 47 wherein the dye is Sybr Green or ethidium bromide.

49. The method of claim 46 wherein a plurality of reaction mixtures having different nucleic acid samples are provided, and the mixtures are concurrently amplified and concurrently detected.

50. A method comprising:
providing a nucleic acid sample and amplification reagents that comprises a first fluorophore attached to a first nucleic acid primer and a second fluorophore attached to a second nucleic acid primer;
amplifying nucleic acid in the sample using the first and second primers; and
at at least a plurality of instances during the amplifying, detecting fluorescence polarization information associated with each of the fluorophores.

51. The method of claim 50 in which the first and second fluorophore have distinguishable absorption and/or emission spectra.

52. The method of claim 51 in which the detecting comprises sequentially detecting fluorescence polarization information of the first fluorophore at a first wavelength and information from the second fluorophore at a second wavelength.

53. The method of claim 50 in which the first and second primer hybridize to the same gene.

54. A method comprising:
providing a nucleic acid sample and amplification reagents that comprises a first fluorophore attached to a first nucleic acid primer, specific for a first nucleic acid species, and a second fluorophore attached to a second nucleic acid primer, specific for a second nucleic acid species;
amplifying nucleic acid in the sample using the first and second primers; and
at at least a plurality of instances during the amplifying, detecting fluorescence polarization information associated with each of the fluorophores.

55. The method of claim 54 in which the detecting comprises sequentially detecting fluorescence polarization information of the first fluorophore at a first wavelength and information from the second fluorophore at a second wavelength.

56. The method of claim 54 in which the first and second fluorophore are selected from the group consisting of: a fluorescein, Texas Red, HEX, Cy3, Cy5, Cy5.5, Pacific Blue, a rhodamine, and Cy7.

57. The method of claim 54 in which at least four different labeled primers are used and detected.

58. An article of machine-readable medium, having embodied thereon instructions that cause a processor to effect a method comprising:
determining intensity values, wherein (i) each intensity value is determined as a function of a value representing fluorescence approximately perpendicular and a value approximately parallel to polarized excitation light, (ii) the fluorescence is detected from a fluorophore attached to a primer specific for a target nucleic acid, and (iii) each intensity value corresponds to an temporal instance during a nucleic acid amplification reaction;
extrapolating an initial intensity value from intensity values within an exponential region of the amplification reaction; and
inferring an initial concentration for the target nucleic acid.

59. The article of claim 58 wherein inferring comprises comparing an inferred initial intensity value for the target nucleic acid to a similarly inferred initial intensity value for a reference nucleic acid of known molecular concentration, and determining an estimated initial molecular concentration for the target nucleic acid.

60. An article of machine-readable medium, having embodied thereon instructions that cause a processor to effect a method comprising:
receiving a plurality of image maps, each map including information about detected light of a defined polarity at a plurality of imaged sites, each imaged site including a primer for amplification of a target nucleic acid; and
determining a value indicative of abundance of extended primers at each of the imaged sites.

61. The article of claim 60 wherein each of the imaged sites corresponds to a sample on a multi-sample carrier.

62. The article of claim 60 wherein the plurality of image maps comprise maps including information about detected light at different instances during a reaction.

63. The article of claim 62 wherein at least one of the imaged sites corresponds to a reference sample of known molecular concentration.

64. The article of claim 63 wherein the instructions further cause a processor to infer an initial concentration of target nucleic acid for at least some of the imaged sites from information for the imaged site that corresponds to a reference sample.

65. A database, stored on machine-readable medium, comprising: data representing (a) fluorescence polarization assessments, (b) reaction samples, (c) temporal information; and associations that relate each fluorescence polarization assessment to a reaction sample and a temporal value.

66. A database, stored on machine-readable medium, comprising: a plurality of image maps, each map including information about detected light of a defined polarity at a plurality of imaged sites, wherein the detected light of each map is associated with a fluorophore and each map is associated with a temporal instance during an nucleic acid amplification reaction.

67. A system comprising:
an apparatus that comprises (1) an amplification control unit that is configured to control reaction conditions at a plurality of sites for nucleic acid amplification; and (2) a fluorescence polarization monitor that is configured to concurrently monitor fluorescence polarization associated with each site of the plurality; and
a processor configured to receive information from the apparatus about the fluorescence polarization and infer initial values that correlate with concentration for a nucleic acid species at each site of the plurality.
